(19) United States
(12) Patent Application Publication
(10) Pub. No.: US 2022/0214480 A1
(43) Pub. Date: Jul. 7, 2022

(54) AIRBORNE CORONAVIRUS DETECTOR AND ALERTING SYSTEM

(71) Applicant: GM GLOBAL TECHNOLOGY OPERATIONS LLC, Detroit, MI (US)

(72) Inventors: Thomas A. SEDER, Fraser, MI (US); Kai-Han CHANG, Madison Heights, MI (US)

(73) Assignee: GM GLOBAL TECHNOLOGY OPERATIONS LLC, Detroit, MI (US)

(21) Appl. No.: 17/142,739

(22) Filed: Jan. 6, 2021

(51) Int. Cl. G02B 5/18; G02B 17/00; G01N 21/41; G01N 33/569; G01N 33/543; B60R 16/023; B60Q 9/00

(52) U.S. Cl. CPC G02B 5/1857; G02B 17/004; G02B 5/1814; B60Q 9/00; G01N 33/56983; G01N 33/54386; B60R 16/023; G01N 21/4133

(57) ABSTRACT

Detecting systems configured to detect airborne viruses, and methods of forming the same, are provide. A method for preparing a system for detecting an airborne virus includes preparing a thin-film coating on a surface of a substrate; ablating the coating to form an optical grating structure; and associating a plurality of receptors having an affinity and specificity for the virus with the optical grating structure so that the structure is capable of indicating the presence of the virus. A detecting system includes the optical grating structure and an optical component. The optical component includes a cavity defined by two opposing reflecting surfaces and configured to receive the optical grating structure, and a light source disposed adjacent to an exterior surface of a first reflecting surface. The light source may be configured to direct light towards the first reflecting surface so that light in resonance enters the cavity.

### INTRODUCTION

This section provides background information related to the present disclosure which is not necessarily prior art.

The severe acute respiratory syndrome coronavirus 2 (SARS-CoV-2) epidemic has changed the way hygiene is managed and maintained in public and other shared spaces. This includes shared spaces, such as passenger compartments in vehicles. SARS-CoV-2, which causes coronavirus disease 2019 (COVID-19), and other deadly microbes can transmit through direct person-to-person contact from the uptake of contaminated airborne or aerosol droplets (e.g., airborne pathway). Such viruses enter the nasal membrane and attach to proteins (such as, angiotensin converting enzymes like angiotensin converting enzyme 2 (“ACE2”)) in humans or other animals that are embedded in cell walls. Surface coatings that render pathogens contaminating surfaces harmless, as well as surface tests are common. However, there exists a lingering need for systems and methods configured for quick detection of airborne viruses.

### SUMMARY

This section provides a general summary of the disclosure, and is not a comprehensive disclosure of its full scope or all of its features.

The present disclosure relates to a detecting system that is configured to detect an airborne virus or viruses (such as, the SARS-CoV-2) and to alert the user(s) of the presence of the airborne virus or viruses.

In various aspects, the present disclosure provides a method for preparing a system for detecting an airborne virus. The method may include preparing a thin-film coating on one or more surfaces of a substrate; ablating the thin-film coating to form an optical grating structure; and associating a plurality of receptors having an affinity and specificity for the airborne virus with the optical grating structure so that the optical grating structure is capable of indicating the presence of the airborne virus.

In one aspect, the associating may include contacting a liquid medium that includes the plurality of receptors with the optical grating structure.

In one aspect, the substrate may be a glass substrate and the thin-film coating may be a self-aligned monolayer that includes a plurality of hydrophobic hydrocarbon tails bonded to a siloxane cross-linked backbone that is disposed parallel with the substrate.

In one aspect, the plurality of receptors may be respectively disposed on distal ends of the hydrocarbon tails oriented away from the substrate, so that each of the plurality of receptors is exposed to a surrounding environment.

In one aspect, preparing the thin-film coating may include contacting the one or more surfaces of the substrate with an organosiloxane precursor.

In one aspect, the receptors may be angiotensin converting enzymes having hydrophobic regions that are associated with the distal ends of hydrophobic hydrocarbon tails.

In one aspect, the ablating may include using a laser holographic technique.

In one aspect, the method may further include disposing the optical grating structure, which includes the plurality of receptors, within an optical cavity defined by two opposing reflecting surfaces.

In one aspect, the optical cavity may be in communication with a photocell detector that is configured to detect changes in at least one of a refractive index and a diffraction efficiency of the grating structure.

In one aspect, the photocell detector may be in communication with an alarm that is configured to emit a signal when a change occurs in the at least one of the refractive index and the diffraction efficiency of the grating structure.

In one aspect, the optical cavity may be in communication with an alarm that is configured to emit a signal when a change occurs in the at least one of a refractive index and a diffraction efficiency of the grating structure.

In various aspects, the present disclosure provides a system for detecting airborne viruses. The system may include an optical grating structure. The optical grating structure may include a substrate; a patterned thin-film coating on one or more surfaces of a substrate; and a plurality of receptors having an affinity and specificity for the airborne virus disposed on the patterned thin-film coating and oriented away from the substrate to be exposed to a surrounding environment.

In one aspect, the substrate may be a glass substrate and the thin-film coating may be a self-aligned monolayer that comprises a plurality of hydrophobic hydrocarbon tails bonded to a siloxane cross-linked backbone that is disposed parallel with the substrate.

In one aspect, the receptors may be angiotensin converting enzymes having hydrophobic regions that are associated with the distal ends of hydrophobic hydrocarbon tails.

In one aspect, the system may further include an optical component. The optical component may include an optical cavity defined by two opposing reflecting surfaces and an incident light source disposed adjacent to an exterior surface of a first reflecting surface. The optical grating structure may be disposed within the optical cavity. The incident light source may be configured to direct light towards the first reflecting surface so that light in resonance enters into the optical cavity.

In one aspect, the system may further include a photocell detector that is in communication with the optical cavity and configured to detect changes in at least one of a refractive index and a diffraction efficiency of the optical grating structure.

In one aspect, the photocell detector may be in communication with an alarm that is configured to emit a signal when a predetermined change occurs in the at least one of the refractive index and the diffraction efficiency of the optical grating structure.

In various aspects, a system for detecting airborne viruses in a passenger compartment of a vehicle. The system may include an optical grating structure and an optical component. The optical grating structure may include a substrate; a patterned thin-film coating on one or more surfaces of a substrate; and a plurality of receptors having an affinity and specificity for the airborne virus disposed on the patterned thin-film coating and oriented away from the substrate to be exposed to a surrounding environment. The optical component may include an optical cavity defined by two opposing reflecting surfaces and an incident light source disposed adjacent to an exterior surface of a first reflecting surface. The optical grating structure may be disposed within the optical cavity. The incident light source may be configured to direct light towards the first reflecting surface so that light in resonance enters into the optical cavity.

In one aspect, the substrate may a glass substrate and the thin-film coating may be a self-aligned monolayer that comprises a plurality of hydrophobic hydrocarbon tails bonded to a siloxane cross-linked backbone that is disposed parallel with the substrate. The receptors may be angiotensin converting enzymes having hydrophobic regions that are associated with the distal ends of hydrophobic hydrocarbon tails.

In one aspect, the system further includes a photocell detector that is in communication with the optical cavity and configured to detect changes in at least one of a refractive index and a diffraction efficiency of the optical grating structure. The photocell detector may be in communication with an alarm that is configured to emit a signal when a predetermined change occurs in the at least one of the refractive index and the diffraction efficiency of the optical grating structure.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIGS. 1A and 1B are schematics of an example airborne virus detection system in accordance with various aspects of the present disclosure;

FIGS. 2A and 2B are schematics of an example of an alert system for an airborne virus detection system in accordance with various aspects of the present disclosure;

FIG. 3 illustrates an example method for forming a system for detecting airborne viruses in accordance with various aspects of the present disclosure;

FIGS. 4A-4C. FIG. 4A is a schematic of a thin film coating on a substrate in accordance with various aspects of the present disclosure. FIG. 4B is a schematic of an ablation process for forming an optical grating structure in a thin-film coating in accordance with various aspects of the present disclosure. FIG. 4C is a schematic of the optical grating structure illustrated in FIG. 3A having a plurality of receptors embedded thereon.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

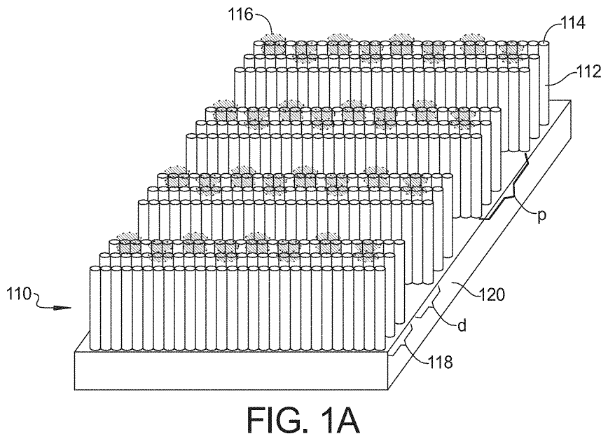

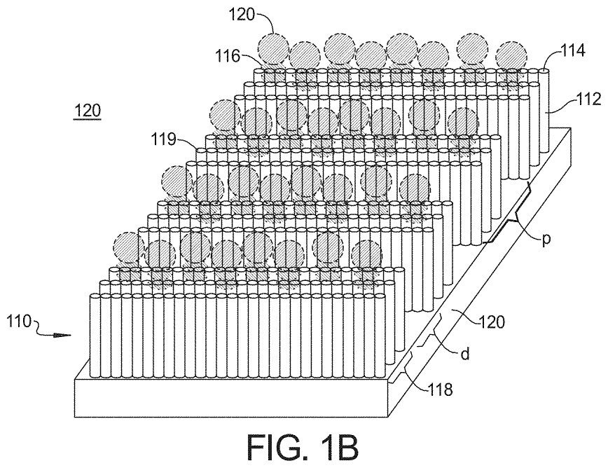

In various aspects, the present disclosure provides a system 100 configured to detect airborne viruses, such as the severe acute respiratory syndrome coronavirus 2 (SARS-CoV-2), present in a surrounding environment. In alternative aspects, it will be appreciated that such a detection system can also be employed with other airborne pathogens. As illustrated in FIG. 1A, the system 100 includes a thin-film coating 110 disposed on one or more surfaces of a substrate 120. The substrate 120 is preferably a thin, substantially flat surface so as to limit or avoid reflective offsets that may degrade performance. In certain instances, the substrate 120 may be transmissive to wavelengths of light generated by a light source. For example, the substrate 120 may be a high-quality glass substrate having a low thermal expansion coefficient and a high optical transmission (e.g., greater than or equal to about 60%) for wavelengths of light generated by a laser diode or other light source. In other instances, the substrate 120 may be a plastic substrate.

The thin-film coating 110 may be a patterned self-aligned monolayer (“SAM”) that includes a plurality of crosslinked hydrocarbon siloxane moieties, including, for example, hydrophobic hydrocarbon tails 112 bonded to a siloxane cross-linked backbone. The siloxane cross-linked backbone may be parallel with the substrate 120, while the hydrophobic hydrocarbon tails 112 may extend from the siloxane cross-linked backbone in an ordered manner. Thus, the thin-film coating 110 may be a one molecule thick layer of material that bonds to a surface of the substrate in an ordered way. In certain instances, as illustrated, the thin-film coating 110 may have a linear pattern that includes a plurality of repeated dimensions (e.g., rows 118) that defines an optical grating, for example a holographic optical element (“HOE”).

An “optical grating structure” typically comprises one or more openings to permit certain wavelength(s) of light to pass through. For example, in certain aspects, an optical grating structure may comprise a plurality of parallel rows 118 or discrete regions spaced apart, but that are substantially parallel to one another. The spacing between adjacent rows defines a plurality of openings through which certain wavelengths of light may pass. Though not illustrated, in certain instances, the grating may also comprise a second plurality of rows having a distinct orientation from the first plurality of rows that are likewise spaced apart, but substantially parallel to one another. The first and second plurality of rows may intersect or contact one another at one or more locations to form a grid or mesh structure.

In certain variations, an optical grating pattern includes rows formed on a surface of the substrate that defines a period “p” (a distance defined from a side of a first row or linear feature to a side of a second adjacent row or linear feature). A distance “d” between adjacent rows is considered an opening (or aperture or slit) through which the target light wavelengths can pass. The thin-film coating 110 may have a pattern that has a grating periodicity (p) of less than or equal to about 2 μm for diffraction angles larger than about 13 degrees. The skilled artisan will appreciate that various other patterns may be formed in the thin film coating 110 to form different optical gratings.

The system 100 may further includes a plurality of receptors 116 disposed on distal ends 114 of the crosslinked hydrocarbon siloxane moieties. More particularly, the plurality of receptors 116 may be disposed on distal ends 114 of the hydrophobic hydrocarbon tails 112, away from the substrate 120. The plurality of receptors 116 have both an affinity and specificity for the targeted airborne virus, such as SARS-CoV-2. Though not illustrated, the skilled artisan will recognize that in certain instances, greater than 99%, for example, 100% coverage of the exposed distal surface 119 of each row 118 is desirable. For example, respective virus receptors 116 may be associated with or coupled to each tail 112. In other instances, however, the desired coverage may be less than 100% (e.g., less than or equal to about 99%, less than or equal to about 95%, less than or equal to about 90%) and other design parameters may be varied accordingly, for example only, a light source input as detailed below. In each instance, the virus receptors 116 may be tailored or selected so as to have an affinity and specificity for the target airborne virus, that is for example, in the present of another virus or viruses the receptors 116 preferentially bind with the targeted virus 120.

In certain variations, the receptors 116 may be angiotensin converting enzymes like angiotensin converting enzyme 2 (“ACE2”). For example, although similar to SARS-CoV, the receptor-binding domain of SARS-CoV-2 differs in several key amino acid residues to permit stronger binding affinity with human ACE2 receptors. Thus, the receptor 116 may be an ACE2 receptor having hydrophobic regions. These hydrophobic regions in the ACE2 receptor 116 may be considered to be tails, that are embedded into the hydrophobic hydrocarbon tails 112 in a manner similar to a cell wall in an animal like a human. When present, an airborne virus or viruses 120 may be associated with (e.g., bound or captured by) the receptors 116, such as illustrated in FIG. 1B. The association of the target virus 120 with the rows 118 in the optical grating thereby causes detectable changes in the refractive index and/or diffraction efficiency of the optical grating, as further discussed below.

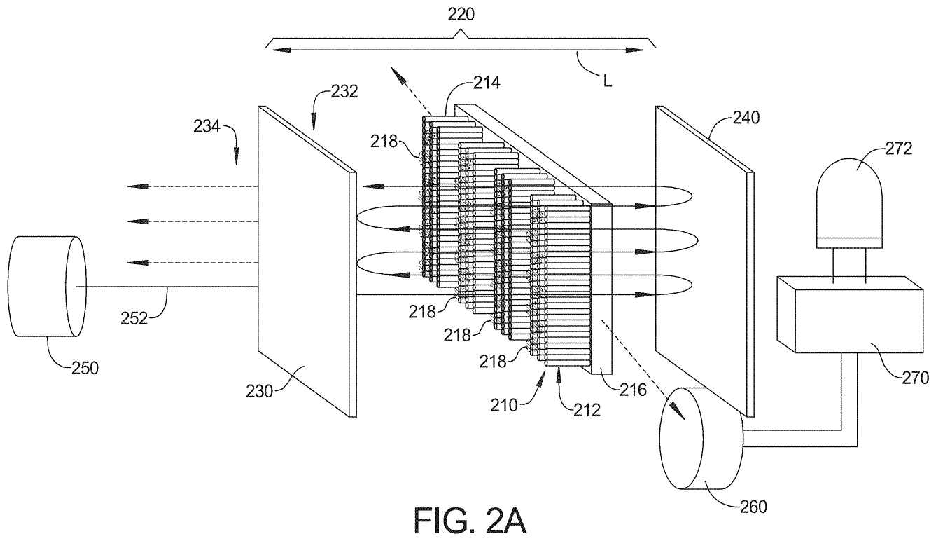

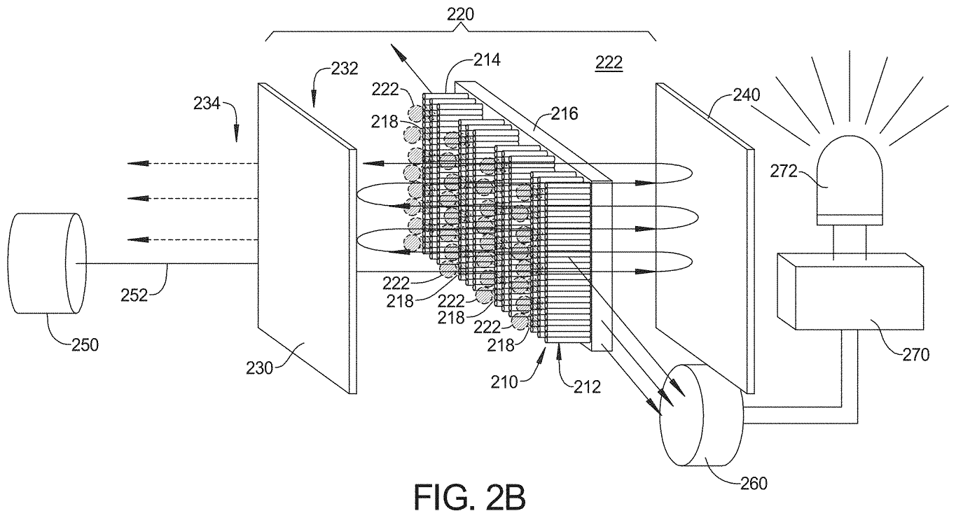

In various aspects, the present disclosure provides a detection system 200 for alerting a user of the presence of an airborne virus or viruses, such as SARS-CoV-2, for example, by providing a signal or alarm that may be detected by a machine or human. As illustrated in FIG. 2A, the alerting system 200 includes a sensing component 210 disposed within an optical cavity 220 defined by two opposing reflective surfaces 230, 240 that reflect wavelengths of light generated by a light source 250, for example, greater than or equal to about 50% of the wavelengths of light generated by the light source 250. As further detailed below, the two opposing reflective surfaces may include a first mirror 230 and a second mirror 240. In certain instances, the first mirror may be a partial reflecting mirror. For example, the reflectivity of the first mirror 230 may be at least about 50%, and the reflectivity of the second mirror 240 may be about 100%, for the wavelength(s) of light 252. In certain instances, the optical cavity 220 may have an average thickness greater than or equal to about 1 μm to less than or equal to about 10 μm.

The sensing component 210 may be a holographic optical element 100 such as illustrated in FIG. 1A. For example, the sensing component 210 may include a thin-film coating 212 disposed on one or more surfaces of a substrate 216. Though not illustrated, in certain instances, one of the first mirror 230 and the second mirror 240 may act as the substrate.

The thin-film coating 212 may be a patterned self-aligned monolayer that includes a plurality of crosslinked hydrocarbon siloxane moieties and plurality of virus receptors 218 disposed on distal ends of the crosslinked hydrocarbon siloxane moieties. More particularly, the plurality of receptors 218 may be disposed on distal ends of the hydrophobic hydrocarbon tails 214, away from the substrate 216. As illustrated, a first side of the sensing component 210 including the tails 214 and the receptors 218 may face a first side 232 of the first mirror 230, while the substrate 216 faces the second mirror 240. The first mirror 230 may be a partial reflecting mirror that allows a portion of light 252 generated by a light source 250 to enter the cavity 220, while reflecting a portion of the light 252 that passes through the sensing component 210. The portion of light 252 that passes within the optical cavity 220 is then reflected internally by the second mirror 240 to generate an optical resonator cavity (e.g., a Fabry-Perot like etalon resonator or interferometer).

The system 200 may further include an incident light source 250, for example, having a wavelength in the visible spectrum, such as a blue (a wavelength in a range of about 435 nm to about 500 nm), green (a wavelength in a range of about 520 nm to about 565 nm), or red (a wavelength in a range of about 625 nm to 740 nm) laser diode or a red vertical cavity surface emitting laser (VCEL). As illustrated, the incident light source 250 may be disposed adjacent to a second side 234 of the first mirror 230 and spaced apart from the detecting component 210. The incident light source 250 generates light 252 that is directed towards the first mirror 230, a portion of which passes through the first mirror 230 and enters into the optical cavity 220. Thus, the portion of light 252 that passes through the first mirror 230 into the optical cavity 220 towards the second mirror 240 is considered in resonance. For example, when the optical cavity 220 thickness is L, the resonance wavelength in the optical cavity 220 may be 2 L/q, where q is an integer. The resonance wavelengths (i.e., able to travel through the first mirror 230 and into the optical cavity 220 and reflected from the second mirror 240) are the modes that can form standing waves within the optical cavity 220. The wavelength of the light source 250 of the detection system should match (e.g., be a multiple of) the resonance wavelength of light 252 of the optical cavity 220, or vice versa, to minimize or prevent intensity loss.

The system 200 may further include a detector to monitor one or more characteristics of the optical cavity 220 and thus to detect the presence of an airborne virus target. When present, an airborne virus or viruses may be bound to or captured by the optical grating and thus cause detectable (e.g., quantifiable) changes in the refractive index change and diffraction efficiency of the optical grating. In certain aspects, the detector may be a photodetector or photocell detector 260. For example, the diffraction of light by the holographic optical element 210 may be detected, for example, by a photocell detector 260 that is sensitive to the wavelength light source 250 and placed at the appropriate angle, for example, off-axis to the holographic optical element 210, as illustrated. The appropriate angle is the diffraction angle of the grating for the light source 250.

When present, an airborne virus or viruses 222 may be captured by the receptors 218, such as illustrated in FIG. 2B, thereby causing detectable changes in the refractive index change and diffraction efficiency change of the holographic optical element 210, for example, by an increase in the amount of blue radiation falling onto the photocell detector 260. The photocell detector 260 may detect the changes in the refractive index and/or diffraction efficiency of the holographic optical element 210. The photocell detector 260 may be in communication with an alarm 270 that is configured to emit a signal for detection by a device or human. In certain aspects, the signal may be sound, light, or other output signal generated when a change in a refractive index and/or diffraction efficiency is recognized by the photocell detector 260. In certain aspects, the output signal may be fed to a processor/computer and generate an alarm in another system, for example, a display system in a vehicle. For example, as illustrated, the alarm 270 may include a light 272. As illustrated in FIG. 2B, when a virus is present the photocell detector 260 detects changes in the refractive index and/or diffraction efficiency of the holographic optical element 210 and the alarm 270 receiving the signal from the photocell detector 260 emits a light so as to alert the user of the presence of the virus. While the systems configured for detecting an airborne virus like SARS-CoV-2 provided by the present technology are particularly suitable for use in passenger compartments of an automobile or other vehicles (e.g., motorcycles, boats, tractors, buses, motorcycles, mobile homes, campers, and tanks) where multiple passengers may be present, they may also be used in a variety of other industries and applications in alternative aspects, such as in buildings, houses, offices, sheds, warehouses, and the like, by way of non-limiting example.

In various aspects, the present disclosure provides a method for forming a system configured to detect airborne viruses, such as the SARS-CoV-2. The method may use molecular imprinting technology (“MIT”). For example, the method generally includes forming a thin film coating having an affinity and specificity for the selected virus, for example the SARS-CoV-2; ablating the thin film coating to remove portions of the film and thus define a grating structure; and disposing a plurality of receptors for the selected virus on exposed surfaces of the grating structure. When present, an airborne virus or viruses may be captured by the receptors thereby causing detectable changes in the refractive index and/or diffraction efficiency of the grating structure.

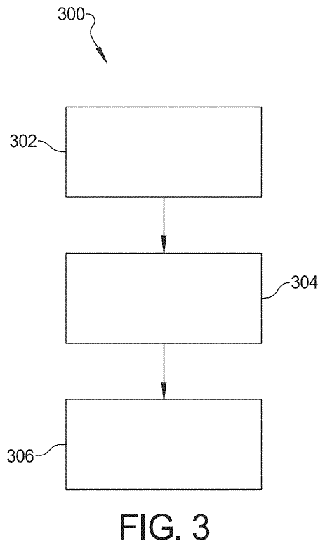

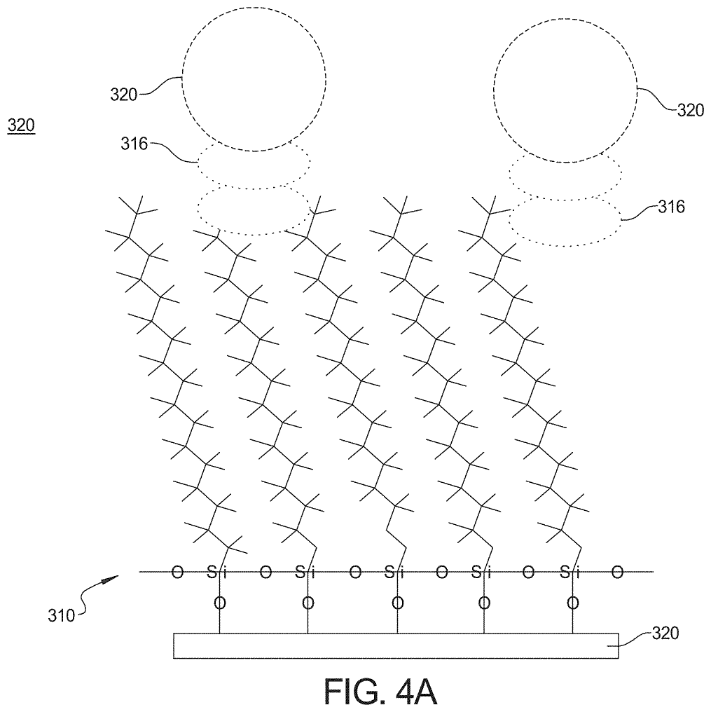

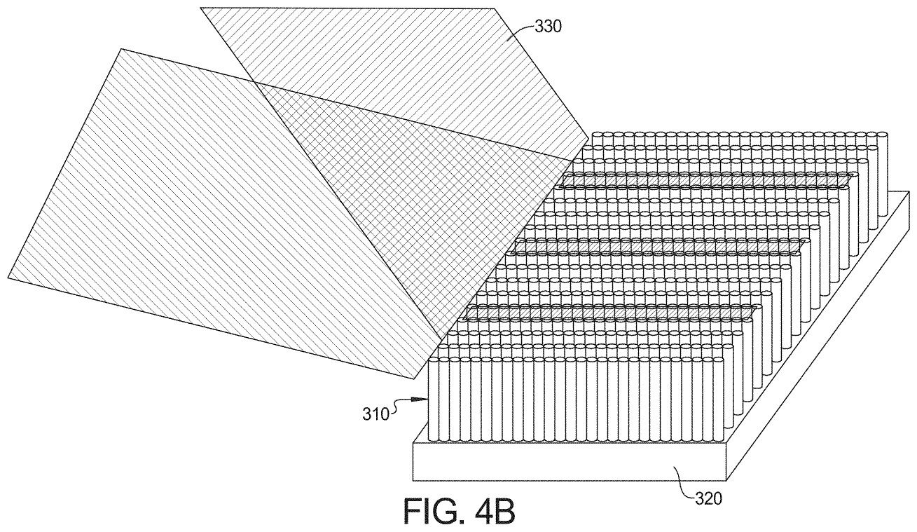

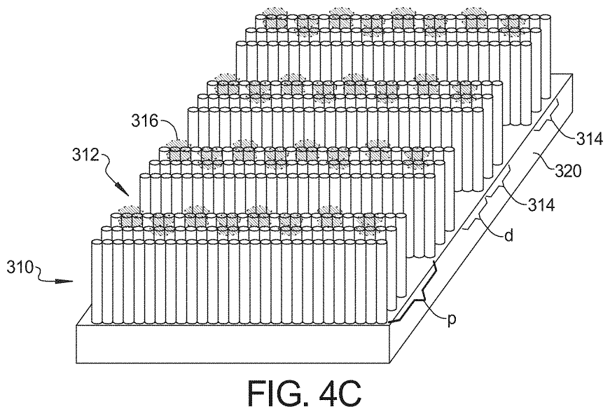

FIG. 3 illustrates an example method 300 for forming a system configured to detect airborne viruses, like system 100 illustrated in FIG. 1A. The method 300 includes preparing 302 a thin film coating 310 on a substrate 320. For example, as illustrated in FIG. 4A, the thin film coating 310 may be a self-aligned monolayer (“SAM”) prepared by contacting a surface of the substrate 320 with an organosiloxane precursor. The organosiloxane precursor forms a hydrocarbon-siloxane crosslinked structure having, for example only, 17 or 19 numbered carbon chain, that defines the thin film coating 310. For example, as illustrated the crosslinking occurs at the silicon-head so as to form a network of —Si—O—Si—O—Si— bonds defining the siloxane. The hydrocarbon moieties or tails are free to float and align with one another so as to minimize the free energy of the system. In this manner, the thin film coating 310 mimics a lipid bilayer of a cell wall. The skilled artisan will appreciate that various other methods may be used to prepare a holographic optical element (“HOE”), including bonding angiotensin converting enzymes directly to a suitable plastic layer of the type used in affinity chromatography for virus isolation.

With renewed reference to FIG. 3, in certain variations, the method 300 may include ablating 304 the thin film coating 310 to remove portions of the thin film coating 310 and thus define a grating structure 312. For example, as illustrated in FIG. 4B, the thin film coating 310 may be ablated using laser holographic techniques, such as ultraviolet laser beam interference 330, during which pulse duration and energy may be modified so as to form the desired pattern. In certain instances, the thin film coating 310 may be ablated using computer generated holography, which may use spatial light modulator with encoded hologram to form desired pattern on thin film coating 310. As illustrated, in FIG. 4C, the grating structure 312 may have a linear pattern that includes a plurality of repeated dimensions (e.g., rows 314) that defines an optical grating, for example a holographic optical element (“HOE”). Though not illustrated, the skilled artisan will appreciate that various other patterns may be formed in the thin film coating 310 to form different optical gratings.

In certain variations, the method 300 may include contacting 306 the grating structure 312 with a liquid medium that includes a plurality of receptors 316. Contacting 306 may include any known method of exposing the grating structure 312 to the receptors 216. For example, contacting 306 may include washing the grating structure 312 with the liquid medium. In certain aspects, the receptors 216 may be dispersed in a suitable liquid medium or solvent that has less affinity for the receptors 216 than the grating structure 312, so that the grating structure 312 (e.g., distal end) extracts the receptor 216 from the liquid. In still other aspects, the receptors 216 may be dissolved in a suitable solvent that solubilizes the receptors 216, but has less affinity for the receptor 216 than the grating structure 312, so that the grating structure 312 extracts the receptor 216 from the liquid. In each instance, the method 300 may further include the liquid from the grating structure 312, leaving the receptors 216 behind.

As illustrated in FIG. 4A, the receptors 316 may be angiotensin converting enzyme 2 (“ACE2”) having a hydrophobic portion that is embedded into the exposed hydrocarbon tails of the grating structure 312, including the hydrocarbon-siloxane crosslinked structure, in a manner similar to a cell wall. When present, the airborne virus or viruses 320 may be associated with, bound to, or otherwise captured by the receptors 316 thereby causing detectable changes in optical grating, such as detectable and optionally quantifiable changes in the refractive index and/or diffraction efficiency of the grating structure, such as further detailed above. For example, when present, the airborne virus or viruses 320 may be associated with the receptors 316 via Van Der Waal force attachment. In certain instances, the coronavirus protein prion (i.e., crown) may be captured by the receptor 316—i.e., the cell wall organelle, the angiotensin converting enzymes.

In various aspects, the present disclosure provides a method for forming a system configured to provide an alert as to the presence of an airborne virus or viruses, such as the SARS-CoV-2. Such a system may be part of a vehicle and may be included in a passenger compartment. The method includes preparing a detector, like system 100 illustrated in FIG. 1A, for example, using method 300 illustrated in FIG. 3, and disposing the detector within an optical cavity defined by two opposing reflecting surfaces, such as illustrated in FIG. 2A.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for preparing a system for detecting an airborne virus, the method comprising:
preparing a thin-film coating on one or more surfaces of a substrate;
ablating the thin-film coating to form an optical grating structure; and
associating a plurality of receptors having an affinity and specificity for the airborne virus with the optical grating structure so that the optical grating structure is capable of indicating the presence of the airborne virus.

2. The method of claim 1, wherein the associating comprises contacting a liquid medium comprising the plurality of receptors with the optical grating structure.

3. The method of claim 1, wherein the substrate is a glass substrate and the thin-film coating is a self-aligned monolayer that comprises a plurality of hydrophobic hydrocarbon tails bonded to a siloxane cross-linked backbone that is disposed parallel with the substrate.

4. The method of claim 3, wherein the plurality of receptors is respectively disposed on distal ends of the hydrocarbon tails oriented away from the substrate, so that each of the plurality of receptors is exposed to a surrounding environment.

5. The method of claim 3, wherein preparing the thin-film coating comprises contacting the one or more surfaces of the substrate with an organosiloxane precursor.

6. The method of claim 3, wherein the receptors are angiotensin converting enzymes having hydrophobic regions that are associated with distal ends of hydrophobic hydrocarbon tails.

7. The method of claim 1, wherein the ablating comprises using a laser holographic technique.

8. The method of claim 1, wherein the method further comprises:
disposing the optical grating structure comprising the plurality of receptors within an optical cavity defined by two opposing reflecting surfaces.

9. The method of claim 8, wherein the optical cavity is in communication with a photocell detector that is configured to detect changes in at least one of a refractive index and a diffraction efficiency of the optical grating structure.

10. The method of claim 9, wherein the photocell detector is in communication with an alarm that is configured to emit a signal when a change occurs in the at least one of the refractive index and the diffraction efficiency of the optical grating structure.

11. The method of claim 8, wherein the optical cavity is in communication with an alarm that is configured to emit a signal when a change occurs in the at least one of a refractive index and a diffraction efficiency of the optical grating structure.

12. A system for detecting an airborne virus, the system comprising:
an optical grating structure comprising:
a substrate;
a patterned thin-film coating on one or more surfaces of a substrate; and
a plurality of receptors having an affinity and specificity for the airborne virus disposed on the patterned thin-film coating and oriented away from the substrate to be exposed to a surrounding environment.

13. The system of claim 12, wherein the substrate is a glass substrate and the thin-film coating is a self-aligned monolayer that comprises a plurality of hydrophobic hydrocarbon tails bonded to a siloxane cross-linked backbone that is disposed parallel with the substrate.

14. The system of claim 12, wherein the receptors are angiotensin converting enzymes having hydrophobic regions that are associated with distal ends of hydrophobic hydrocarbon tails.

15. The system of claim 12, further comprising:
an optical component, the optical component comprising:
an optical cavity defined by two opposing reflecting surfaces, wherein the optical grating structure is disposed within the optical cavity; and
an incident light source disposed adjacent to an exterior surface of a first reflecting surface, wherein the incident light source is configured to direct light towards the first reflecting surface so that light in resonance enters into the optical cavity.

16. The system of claim 15, further comprising:
a photocell detector that is in communication with the optical cavity and configured to detect changes in at least one of a refractive index and a diffraction efficiency of the optical grating structure.

17. The system of claim 16, wherein the photocell detector is in communication with an alarm that is configured to emit a signal when a predetermined change occurs in the at least one of the refractive index and the diffraction efficiency of the optical grating structure.

18. A system for detecting an airborne virus in a passenger compartment of a vehicle, the system comprising:
an optical grating structure comprising:
a substrate;
a patterned thin-film coating on one or more surfaces of a substrate; and
a plurality of receptors having an affinity and specificity for the airborne virus disposed on the patterned thin-film coating and oriented away from the substrate to be exposed to a surrounding environment; and
an optical component, the optical component comprising:
an optical cavity defined by two opposing reflecting surfaces, wherein the optical grating structure is disposed within the optical cavity; and
an incident light source disposed adjacent to an exterior surface of a first reflecting surface, wherein the incident light source is configured to direct light towards the first reflecting surface so that light in resonance enters into the optical cavity.

19. The system of claim 18, wherein the substrate is a glass substrate and the thin-film coating is a self-aligned monolayer that comprises a plurality of hydrophobic hydrocarbon tails bonded to a siloxane cross-linked backbone that is disposed parallel with the substrate, and
wherein the airborne virus is severe acute respiratory syndrome coronavirus 2 (SARS-CoV-2) and the receptors are angiotensin converting enzymes having hydrophobic regions that are associated with distal ends of hydrophobic hydrocarbon tails.

20. The system of claim 18, further comprising:
a photocell detector that is in communication with the optical cavity and configured to detect changes in at least one of a refractive index and a diffraction efficiency of the optical grating structure,
wherein the photocell detector is in communication with an alarm that is configured to emit a signal when a predetermined change occurs in the at least one of the refractive index and the diffraction efficiency of the optical grating structure.
